(19) United States
(12) Patent Application Publication
(10) Pub. No.: US 2006/0089589 A1
(43) Pub. Date: Apr. 27, 2006

(54) Resistive shunt valve

(76) Inventor: Harold D. Portnoy, West Bloomfield, MI (US)

Correspondence Address: Robert P. Renke, Suite 250, 28333 Telegraph Road, Southfield, MI, 48034, US

(21) Appl. No.: 11/256,252

(22) Filed: Oct. 21, 2005

Related U.S. Application Data
(60) Provisional application No. 60/620,945, filed on Oct. 21, 2004.

(51) Int. Cl. A61M 5/00

(52) U.S. Cl. 604/9

(57) ABSTRACT

A resistive shunt valve (RSV) for draining cerebrospinal fluid. The RSV is comprised of a conduit housing and a valve member. The conduit housing is adapted for continuously conveying a flow of cerebrospinal fluid (CSF) with the valve member metering the flow of CSF therethrough. The valve member has a plurality of balls and at least one valve seat, with the number of balls being greater than the number of valve seats. The balls register within the valve seats in the direction of CSF flow. Small notches or irregularities are provided in the valve seats. Metering of CSF can be controlled by the housing diameter, ball diameter, number of balls, ball weight, irregularity of the valve seats, surface characteristics of the ball, and ball material.

### CROSS REFERENCE TO RELATED APPLICATION

The present application claims priority from U.S. Provisional Application Ser. No. 60/620,945 filed Oct. 21, 2004, and entitled “Resistive Valve For Preventing Overdrainage In A Shunt For Hydrocephalus.”

### TECHNICAL FIELD

The present invention relates generally to hydrocephalus, and more particularly to a resistive shunt valve having a simple and robust construction to prevent overdrainage of cerebrospinal fluid.

### BACKGROUND

Shunts play a pivotal role in preventing damage to the nervous systems of hydrocephalic patients. For years, shunts have been designed with the goal of properly draining the cerebrospinal fluid (CSF) accumulating within the cerebral ventricles to regulate intracranial pressure (ICP).

A shunt for hydrocephalus treatment typically includes an intracranial catheter connected to a valve that regulates flow of CSF, and a distal catheter to a cavity within the patient's body. In general, the cavity is the peritoneum or the right auricle of the heart. Shunts are intended to be lifelong implants and must faithfully regulate ICP for all body positions. At the same time, shunt designs should compensate for sudden negative pressures in the hanging distal catheter when the patient goes from a lying position to a sitting or standing position (verticalization).

To date, most shunt designs have suffered from overdrainage tendencies. Overdraining CSF can present headaches, nausea, subdural hematoma, premature closure of cranial sutures, repeated shunt obstruction and other undesirable symptoms in a shunted patient. These shunts have typically incorporated a differential pressure valve. Differential pressure valves have taken the form of proximal or distal slit designs, diaphragms, or ball-seat constructions. Most differential pressure valves adequately drain in the horizontal patient position, but overdrain in the upright patient position. In some cases differential pressure valve shunt designs underdrain in the horizontal position, and overdrain in the vertical position.

There are two general classes of valves designed to prevent overdrainage of CSF. One class of shunts uses the increasing weight of the water column in the hanging distal catheter during verticalization to manipulate a diaphragm to increase the shunt resistance. These are commonly referred to as anti-siphon valves. The major shortcoming of the anti-siphon type valves is that the diaphragm may become encapsulated by scar tissue and cause the valve to fail either in a fully open or closed position.

The other major class incorporates the various gravitational valves in which an element, usually a ball, causes the valve to switch from a low to a high resistance to CSF flow. Several gravitational valve designs have been developed to address overdrainage. Typically, the gravitational valve augments the performance of the differential pressure valve. One known shunt valve system has a compound construction comprised of a high resistance valve and a low resistance valve in serial connection. The low resistance valve typically includes a cone-shaped seat, a ball for sealingly engaging the seat, and a spring that forces the ball against the seat. The low resistance valve typically meters the flow of CSF when the patient is in a supine position, an upright position, and any other position therebetween. The high resistance valve includes one or more stacked balls for sealingly engaging the lowermost ball to a cone-shaped seat against the flow of CSF when the patient is in the upright position. The balls have a predetermined total mass for overcoming the hydrostatic pressure created by the flow of CSF.

One drawback of this shunt valve system is the difficulty in determining a sufficient total ball mass in view of the varying hydrostatic pressure that is caused by the constant movement of the distal catheter in the peritoneal cavity. In addition, this system can completely block the flow of CSF and thus may not sufficiently drain CSF under certain circumstances. A further drawback is created when the valve is not implanted along the long axis of the patient. In this instance, the effective weight of the balls is reduced and allows overdrainage.

Another gravitational system provides only one ball for engaging a single valve seat in the direction of CSF flow. Again, this device suffers from the disadvantage of readily unseating when the device is positioned at any angle other than absolute vertical. Because no device can be implanted in an absolute vertical position, and patient posture constantly changes, this valve design likewise lacks the ability to adequately control the leak rate of CSF to maintain desired ICP.

It would, therefore, be desirable to provide a simple and robust resistive shunt valve with verticalization-dependent efficacy for decreasing ICP without adversely underdraining or overdraining CSF.

### SUMMARY OF THE INVENTION

A resistive shunt valve (RSV) for preventing overdrainage of cerebrospinal fluid (CSF) is provided. The RSV comprises a conduit housing and a valve member. The conduit housing is adapted for continuously conveying a flow of CSF with the valve member metering the flow rate of CSF. The conduit housing and the valve member are adapted for minimizing occlusion of the valve by CSF debris. Further, the housing and the valve member are adapted to minimize overdrainage of CSF.

The housing is sized to hold a plurality of small balls which, when registered in a valve seat or seats act to restrict the flow of CSF through the housing. The balls are seated in the direction of flow created by the suction effect. Preferably, there are more ball members than valve seats in order to provide a heavier mass and geometric configuration necessary to dislodge a ball from its corresponding valve seat under controlled circumstances. A number of factors dictate the relative ease with which the seal created by the ball and seat can be broken. These include the number, weight, composition and size of the balls; the valve seat design; and chamber design. By varying these parameters, the inventive resistive shunt valve can be optimized for various effective lengths of the distal catheter and orientation of the implanted valve, and, therefore, for varying patient needs. To prevent complete flow restriction, irregularities such as small notches are provided in the valve seats in order to allow a restricted flow of CSF when ball members are positioned in each of the valve seats.

In one embodiment, a resistive shunt valve is provided having a housing defining an axis and adapted for continuously conveying a flow of cerebrospinal fluid from an inlet to an outlet. The RSV includes a valve having at least one valve seat adjacent the outlet and at least two balls. The balls are all the same size, and are adapted to register within a corresponding valve seat in the direction of cerebrospinal fluid flow. The number of balls is greater than the number of valve seats, and each of the balls or seats includes an irregularity forming a leaky ball-in-seat seal. The valve adapted to prevent overdrainage of cerebrospinal fluid at a predetermined angle from a vertical orientation of the axis. In one example, the RSV has three valve seats and at least four balls. The housing can also be a straight-walled cylinder. Alternatively, the housing can be cylindrical and include a widened body portion defined by an annular wall set at an angle with respect to the axis. This angle can be between 15 and 75 degrees from the axis. To keep the balls within the housing, the inlet has a diameter less than a ball diameter. Further, the leaky valve can be formed by the balls having a non-spherical or scored surface. Alternatively, or additionally, the valve seats can have at least one notch or rib, or be non-circular. In another aspect, balls occupy between approximately 20-50 percent of the housing volume. In yet a further aspect, a seat diameter (d) to ball diameter (D) ratio is between approximately 0.25-0.75.

In another embodiment, a RSV is provided which includes a housing defining an axis and adapted for continuously conveying a flow of cerebrospinal fluid from an inlet to an outlet, and a gravitational valve having one valve seat adjacent the outlet and at least two balls. One of the at least two balls is adapted to register within the valve seat in the direction of cerebrospinal fluid flow. The valve creates a high resistance position for conveying CSF flow when the axis is in a vertical orientation, and a low resistance position for conveying CSF flow at a predetermined angle from the vertical orientation. The valve includes an irregularity forming a leaky seal providing a maximum flow rate of 0.3-0.5 ml/min in the high resistance position.

In a further embodiment, a RSV for metering flow of cerebrospinal fluid is provided which includes a housing defining an axis and having an inlet and an outlet; a plurality of balls, each the same size; and at least two valve seats adjacent the outlet and each adapted to receive one of the plurality of balls in the direction of cerebrospinal fluid flow to form a leaky valve. The number of balls is greater than the number of valve seats, and each leaky valve is adapted to provide a high resistance flow rate between a vertical orientation of the axis and a predetermined angle from the vertical orientation of the axis.

One advantage of the claimed invention is that it provides a simple and robust resistive shunt valve with verticalization-dependent efficacy for preventing the overdrainage of CSF.

Another advantage of the claimed invention is that an RSV is provided that has a stable and robust construction that reduces CSF debris from clogging the RSV. The multi-ball design, which widely opens the valve seat in the fully open position, permits the balls to re-register, thereby providing a self-cleaning action and a different ball surface for registering within the valve seat during each cycle.

Yet another advantage of the claimed invention is that an RSV is provided that has a simple and compact structure that can be readily manufactured without high-precision manufacturing processes which are typically associated with increased cost of production.

The features, functions, and advantages can be achieved independently and in various embodiments of the present invention or may be combined in yet other embodiments.

### BRIEF DESCRIPTION OF THE DRAWINGS

For a more complete understanding of this invention, reference should now be made to the embodiments illustrated in greater detail in the accompanying drawings and described below by way of examples of the invention:

FIG. 1 is a cross-sectional view of a resistive shunt valve in a supine position, according to one embodiment of the claimed invention;

FIG. 2 is a cross-sectional view of the resistive shunt valve shown in FIG. 1, illustrating the resistive shunt valve in an upright position;

FIG. 3 is an enlarged view of the resistive shunt valve shown in FIG. 2;

FIG. 4 is a cross-sectional view of the resistive shunt valve shown in FIG. 3, as taken along line 4-4, without the balls;

FIGS. 5A-5C show details of alternative ball and valve seat constructions;

FIG. 6 is a cross-sectional view of a resistive shunt valve according to yet another embodiment of the claimed invention;

FIG. 7 is a cross-sectional view of the resistive shunt valve shown in FIG. 6, illustrating the resistive shunt valve in an upright position;

FIG. 8 is an enlarged view of the resistive shunt valve shown in FIG. 7;

FIG. 9A is a cross-sectional view of the resistive shunt valve shown in FIG. 8, as taken along line 8-8;

FIG. 9B is a cross-sectional view of a resistive shunt valve similar to FIG. 9A, in an alternative embodiment; and

FIG. 10 is a cross-sectional view of a resistive shunt valve according to another embodiment of the claimed invention;

FIG. 11 is a cross-sectional view of the resistive shunt valve shown in FIG. 10, illustrating the resistive shunt valve in an upright position;

FIG. 12 is a schematic view of a resistive shunt valve according to the present invention implanted within a hydrocephalic patient.

### DETAILED DESCRIPTION

In the following figures, the same reference numerals are used to identify the same or similar components in the various representative views.

The present invention is particularly suited for a resistive shunt valve for preventing overdrainage of cerebrospinal fluid (CSF) for a hydrocephalic patient where the valve in the vertical position should not drain CSF at more than the rate of approximately 0.3-0.5 ml/min. In this regard, the embodiments described herein employ features where the context permits, e.g. when a specific result or advantage of the claimed invention is desired. However, it is contemplated that the resistive shunt valve can instead be utilized for hydraulic systems or a variety of other suitable applications. To that end, a variety of other embodiments are contemplated having different combinations of the described features, having features other than those described herein, or even lacking one or more of those features.

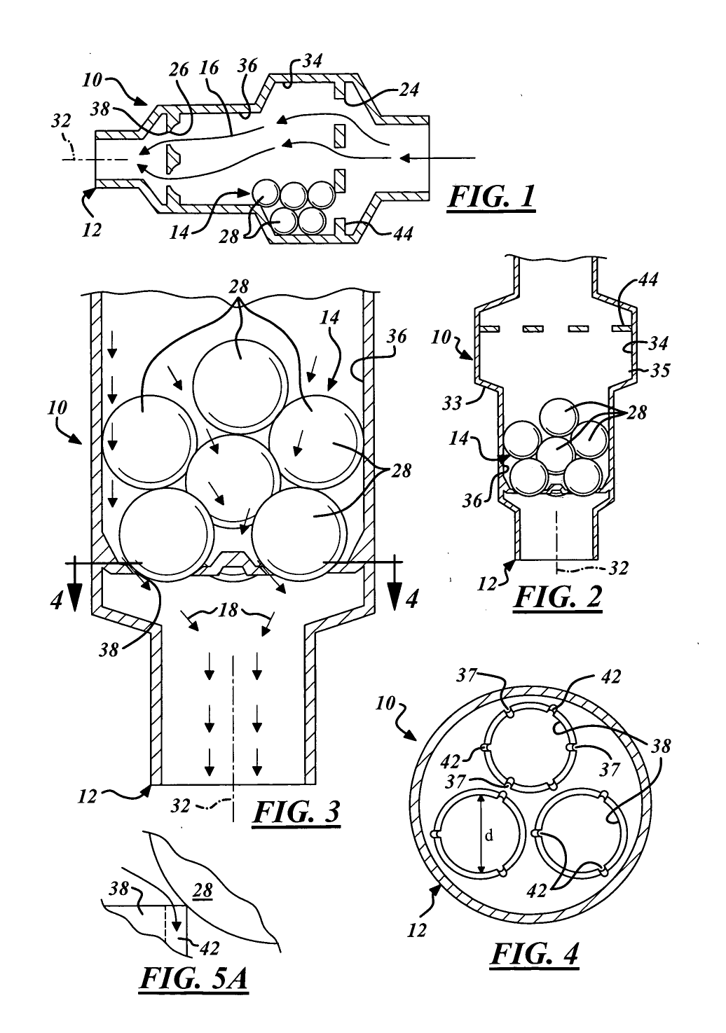

An embodiment of a resistive shunt valve according to the invention is shown in FIGS. 1-4 and referred to generally by the reference numeral 10. FIGS. 1 and 2 are cross-section views of an inventive resistive shunt valve (RSV) in the supine and upright position, respectively. These are the extreme positions where the RSV would be oriented as implanted in a patient with the valve oriented in the longitudinal axis of the patient, when the patient was lying down or standing/sitting upright. FIG. 3 is an enlarged view of the resistive shunt valve shown in FIG. 2. FIG. 4 is a cross-sectional view of the resistive shunt valve shown in FIG. 3, as taken along line 4-4, without illustrating the balls.

The RSV has a conduit housing 12 and a valve member 14 for continually conveying cerebrospinal fluid (CSF) through the housing 12. The valve members 14 comprise a plurality of balls 28 and a plurality of valve seats 38. The number of balls 28 is greater than the number of valve seats 38. In the example of FIGS. 1-4, seven balls 28 (six of which are visible) and three valve seats 38 are illustrated, although it is to be understood that in accordance with this embodiment more balls and valve seats can be provided, so long as the number of balls is greater than the number of valve seats. For multi-ball embodiments, the number of balls 28 will range between 3-20, and the number of valve seats 38 will typically range between 2-5. Thus, in accordance with another embodiment, four balls 28 and three valve seats 38 are provided.

Without limiting the invention, the housing 12 has a chamber 36 which is about 3-6 mm in diameter and is approximately 10-15 mm in length. The chamber 34 defines a cavity 35. The chamber 34 can be straight-walled, or include a widened body portion 34, as shown. The widened portion 34 provides additional volume for the balls 28 to settle in when full CSF flow is desired. The configuration of the widened portion 34 can also be adapted to control the rate at which the balls 28 entrain toward the valve seats 38 under the influence of gravity and/or the suction effect during verticalization. For example, the angle and depth of the annular wall 33 defining the widened portion 34 will influence the reaction of the balls 28 under influence of gravity and/or the suction effect, particularly if the widened portion 34 is closer to the seats 38. A more perpendicular annular wall 33 and a deeper annular wall 33, will restrict the freedom with which the balls 28 move toward the valve seats 38. Conversely, a shallow and/or angled annular wall 33, as shown, will permit somewhat unrestricted freedom of movement for the balls 28. Typically, the annular wall will be set between 15 and 75 degrees from vertical. Of course, a straight-walled chamber 34 (zero degrees from vertical) would provide the least resistive ball entrainment. The housing 12 also may include a screen 44 toward the inlet or proximal end to prevent the balls 28 from entering the connecting catheter. So long as the inlet to the housing 12 is smaller in diameter than the ball diameter, however, the balls 28 will remain within the device. In such instances, the screen 44 may be omitted.

The housing 12 can have any configuration, but preferably has a circular cross-section. The housing can be made from a moldable biocompatible plastic material, but it could also be made from a non-magnetic metal material, such as titanium or the like. The size and shape of the housing is determined to provide desired CSF flow rate in various patient positions.

The balls can be approximately 0.3-2.0 mm in diameter, but typically will be in the range of approximately 0.75-1.5 mm in diameter. The size of the chamber 36 and balls 28, as well as the number of balls can be determined depending on many factors, including the age and height of the patient, as these factors may influence the effective length of the distal catheter, and the corresponding strength of the suction effect created during verticalization by the patient. The balls 28 may be made from a lightweight, non-toxic, non-biodegradable material such as titanium or tantalum. The balls may also be made of a glass or jewel-type material, such as rubies or sapphires. The balls 28 further may be coated with a ceramic, plastic or other suitable material to reduce their surface friction and aid in re-registering. For example, they may be Teflon-coated. The balls 28 can have a smooth surface to readily mate with a corresponding valve seat 38, or an imperfect or scored surface to provide a leaky valve seat. Preferably, all balls within the device are the same size. The balls 28 will typically occupy approximately 20-50 percent of the volume of the chamber 36.

Because multiple balls are used, and the balls only occupy a percentage of the chamber volume, each time the patient changes from the upright position to the supine position, the balls 28 randomly fall away. This acts as a self-cleaning mechanism should CFS debris be present. Further, because the balls are reindexed, they present a different surface to the valve seat upon the next registration cycle.

In another aspect, the balls 28 can be made of different materials to have differing weights and thereby influence the reaction characteristics of the RSV. Lighter balls 28 would entrain more readily and be more likely to register within a valve seat under the influence of rapid CSF flow. Heavier balls would react more slowly to CSF flow, and be more readily influenced by gravity. In either case, however, the additional balls aid in maintaining the seated balls in register with a corresponding valve seat.

Each of the valve seats 38, preferably have one or more notches or slots 42 which allow some seepage or drainage of CSF through the RSV even when one of the balls 28 is situated in each of the valve seats 38, i.e., a leaky valve seat. The notches 42 can be a few thousandths of an inch in width. It is also contemplated to provide a leaky valve seat by modifying the seat geometry such that a ball imperfectly registers within the seat and, thereby allows fluid to pass. This can be accomplished by, for example, providing at least one rib 37 or other protuberance in the valve seat 38 to create a leaky seal. The valve seats 38 can also be non-circular to provide the desired leak rate. Alternatively, slightly non-spherical balls could be used to provide the desired leaky seal.

The additional balls (i.e. at least one more than the number of valve seats) adds additional weight to the balls seated within the valve seats, thereby making them less likely to readily unseat during patient movement from a vertical to an inclined or lying position. Thus, the number of balls to valve seats can influence the angle at which the balls unseat. The chamber design can likewise be designed to vary the angle at which the balls unseat from the valve seats. Also, the smaller the valve seats relative to ball size, the less the force or angle of orientation is needed to unseat the balls and open the valve. Furthermore, in a multi-seated configuration, the valve seats 38 need not all be of the same size such that the ball in one valve seat is more easily dislodged than the others.

Because multiple balls and valve seats are employed to prevent overdrainage, the flow rate versus device angle can be modified. In this example, three valve seats are provided. Thus, the number of balls and the chamber design will dictate the angle at which any one of the valve seats begins to open, i.e., one of the seated balls begins to break its seat seal. Additionally, the device symmetry provides more robust performance as compared to single valve seat designs. Unlike single valve seat—single ball designs which are susceptible to breaking the ball-valve seat seal at any angle other than absolute vertical, the present design controls the angle from the vertical at which the balls unseat.

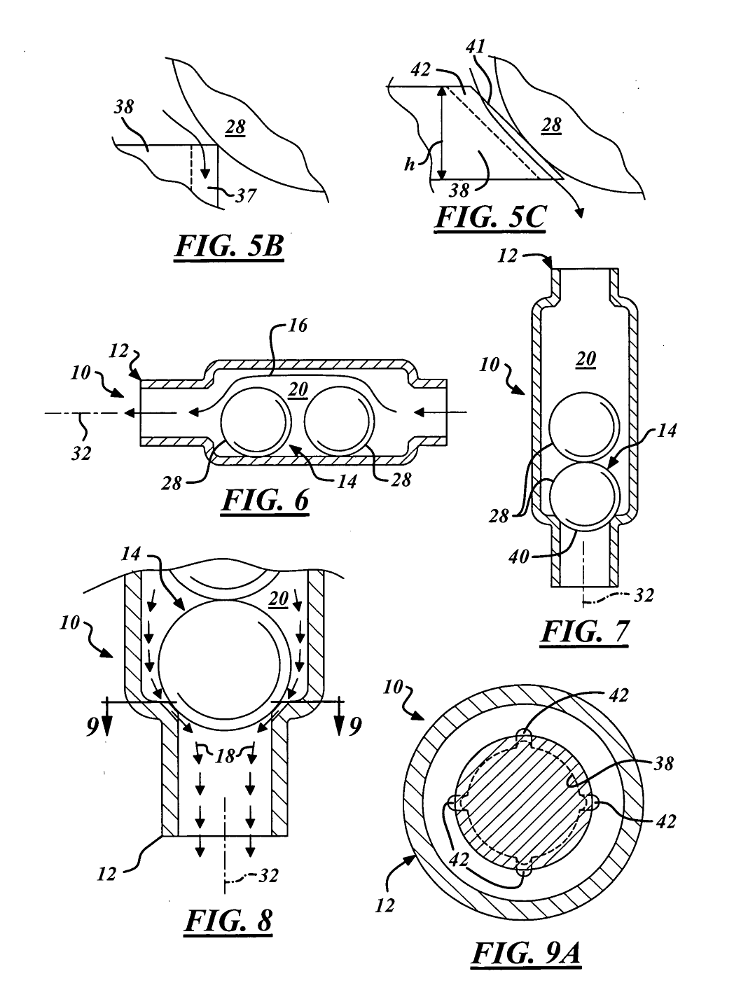

FIGS. 5A-5C show several embodiments for the ball and valve seat constructions. FIG. 5A shows the valve seat 38 with a notch 42 which permits restricted flow of CSF (indicated by the arrow) while a ball 28 is in register with the valve seat 38. FIG. 5B shows a rib 37 which prevents the ball 28 from completely sealingly engaging the valve seat 38, thereby permitting restricted flow of CSF (indicated by the arrow) while a bail 28 is in register with the valve seat 38.

FIG. 5C shows a modified valve seat geometry to aid in maintaining the ball 28 in register with the valve seat 38. In this example, the height (h) of the valve seat wall in combination with the conformity of the seating surface 41 to the ball 28 provides an opportunity to modify the release characteristics of the ball 28 with respect to the valve seat 38. In this regard, the seating surface 41 may be considered a variable diameter (d) valve seat 38.

The release characteristics of a ball 28 registered within valve seat 38 will be determined by the relationship between the ball diameter D and the seating surface diameter d for valve seat designs such as shown in FIGS. 5A and 5B, as well as the geometry of the valve seat for designs such as discussed with respect to FIG. 5C. When the ratio of the seat diameter to the ball diameter falls below, for example, 0.20, the registered ball will more readily unseat. Conversely, as d/D approaches 0.99, the registered ball may not unseat until a nearly horizontal shunt position is obtained. Typically, the ratio will be in the range of approximately 0.25-0.75. The weight of the ball in register, and the force applied by the additional balls will also influence the release profile of the shunt valve. Correspondingly, the chamber design, as discussed above, will also influence how readily the additional balls fall away from the seated balls.

In operation, the valve member 14 provides a clearing position (shown in FIG. 1) and an obstructing position (shown in FIG. 2), as well as variable resistance characteristic for positions in between. The RSV provides a low resistance path 16 for CSF when it is oriented in the supine position shown in FIG. 1 and the balls are unseated from the valve seats. The RSV provides a high resistance path for CSF when it is oriented in the vertical position shown in FIG. 2 and one of the balls is seated in each of the valve seats and the extra balls are positioned on top of the seated balls.

Referring now to the embodiment shown in FIGS. 6 through 9, the conduit housing 12 has a single shell construction defining a chamber 20 with two or more balls 28 contained therein. Although only two balls 28 are shown, three or more balls can be provided in the chamber depending on the desired force needed to open and close the valve. The more balls present, the heavier the stack of material (i.e. ball mass) which has to be displaced in order to unseat the registered ball and fully open the valve. Thus, the stack of balls help determine the angle relative to vertical that the ball-valve seat seal will start to release. The balls 28 register within the valve seat 38 in the direction of CSF flow.

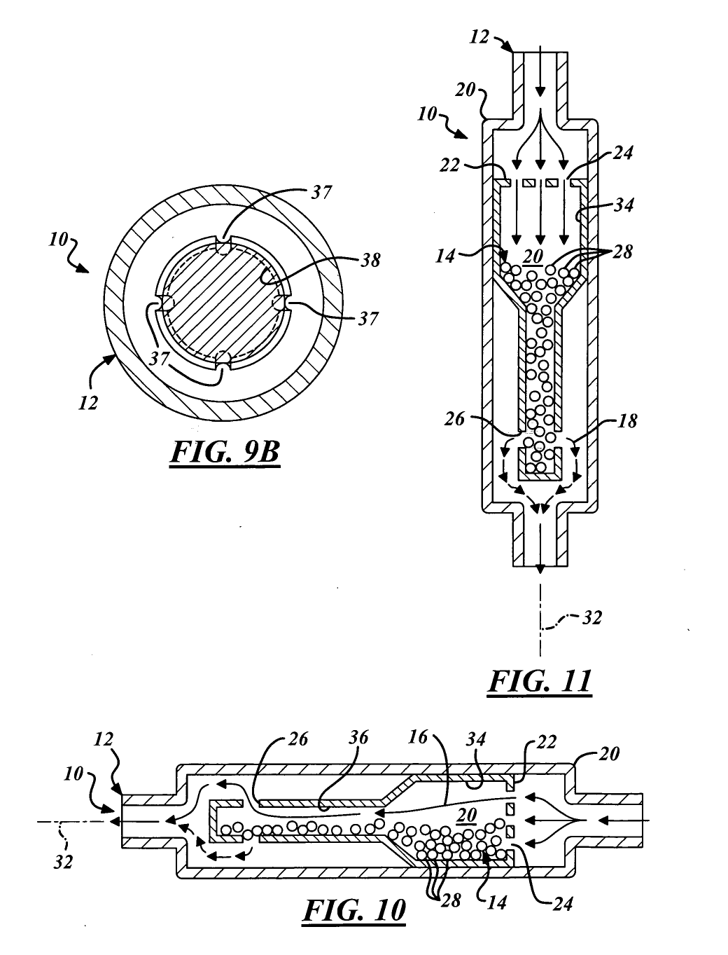

In this embodiment, as best shown in FIG. 6, the chamber 20 is sufficiently large for moving the ball 28 to the clearing position without substantially obstructing the flow of CSF. In this way, the RSV 10 provides the low resistance path 16 for the CSF and the maximum flow rate associated therewith. As shown in FIGS. 7 and 8, the ball 28 is movable to the obstructing position for substantially obstructing the flow of CSF and providing the high resistance path 18. Namely, the housing 12 defines an outlet aperture 40 and a valve seat 38 adjacent thereto that has one or more notches 42. These notches 42 form a gap between the seat 38 and the ball 28 for conveying CSF through the RSV 10, i.e., a leaky valve. The construction of the valve seat 38 can be as described with respect to FIGS. 5A-5C. Thus, in addition or in the alternative, it may include notches 42 (FIG. 9A), ribs 37 (FIG. 9B), or a variable diameter valve seat as previously discussed. In the embodiment shown, no screen is necessary if the ball diameter is larger than the inlet opening at the proximal end of the shunt.

Again, the weight of the ball 28 and its geometry with respect to the valve seat 38 will dictate the drainage characteristics of the RSV. Similarly, in this example too, the release profile of the ball, once registered, can be influenced by the chamber design. For instance, a ball diameter D to chamber diameter ratio greater than 0.50 will unlikely provide completely unrestricted flow. Similarly, as the ratio approaches 0.90, flow will be restricted in all positions, and seat-break may not completely occur until a nearly horizontal valve orientation is presented.

The balls 28 may be made from a lightweight, non-toxic, non-biodegradable material such as titanium or tantalum. The balls may also be made of a glass or jewel-type material, such as rubies or sapphires. The balls 28 further may be coated with a ceramic, plastic or other suitable material to reduce their surface friction and aid in re-registering. For example, they may be Teflon-coated. The balls 28 can have a smooth surface to readily mate with a corresponding valve seat 38, or an imperfect or scored surface to provide a leaky valve seat. When a closely fit chamber design is used, the ball intended to register with the valve seat can differ from the other balls as the other balls will only act as weights for the registered ball. Thus, the additional balls could be a different size and/or different material.

Again, because multiple balls are used, and the balls only occupy a percentage of the chamber volume, each time the patient changes from the upright position to the supine position, the balls 28 fall away from the valve seat 38. This acts as a self-cleaning mechanism should CFS debris be present. Further, because the balls are reindexed, they present a different surface to the valve seat upon the next registration.

Referring to FIGS. 10 and 11, there are shown cross-sectional views of a resistive shunt valve 10 (RSV) respectively in supine and upright positions. Namely, FIGS. 10 and 11 respectively show the configuration of the RSV 10 when a patient having the RSV 10 implanted therein is in a supine position and an upright position.

The RSV 10 has a simple and stable construction for continuously draining cerebrospinal fluid (CSF) without overdraining or underdraining CSF. Also, the RSV 10 regulates the flow of CFS without becoming occluded by CFS debris.

With attention to FIG. 10, the RSV 10 is comprised of a conduit housing 12 and a valve member 14 for continuously conveying CSF through the conduit housing 12. The valve member 14 is movable between a clearing position (shown in FIG. 10) and an obstructing position (shown in FIG. 11). The RSV 10 provides a low resistance path 16 for CSF when the valve member 14 is moved to the clearing position and a high resistance path 18 when the valve member 14 is moved to the obstructing position.

The conduit housing 12 is comprised of an outer shell 20 and an inner shell 22. In this embodiment, the outer shell 20 and the inner shell 22 are comprised of a moldable biocompatible plastic. It will be appreciated that the outer shell 20 and the inner shell 22 can instead be comprised of a non-magnetic metal, titanium, a metal alloy, other suitable materials, or any combination thereof. Also, in this embodiment, the inner shell 22 defines a series of inlet orifices 24 and outlet orifices 26, which are sized for metering a flow of CSF therethrough. It is understood that the inner shell 22 and/or the outer shell 20 can have orifices 24, 26 that are sized and shaped for providing a predetermined flow rate and a predetermined flow pattern.

As shown in FIG. 10, the inlet orifices 24 and the outlet orifices 26 determine the maximum flow rate when the valve member 14 is moved to the clearing position. In other words, orifices 24, 26 meter the flow of CFS and provide the low resistance path 16 for CSF when the valve member 14 is moved to the clearing position.

With attention now to FIG. 11, the valve member 14 in the obstructing position, in conjunction with the orifices 24, 26, substantially obstruct the flow of CSF and accordingly provide the high resistance path 18 for producing a minimum flow rate. In this embodiment, the valve member 14 is comprised of a plurality of balls 28 sized larger than the orifices 24, 26 in the inner shell 22 so as to retain the balls 28 within the RSV 10.

The inner shell 22 is configured for sufficiently moving the balls 28 therein to obstruct the flow of CSF when the RSV 10 is in the upright position. Namely, the inner shell 22 defines a primary chamber 30 with the balls 28 contained therein. The volume of the primary chamber 30 decreases along a longitudinal axis 32 of the housing 12 in the direction of the CSF flow.

In this embodiment, the inner shell 22 defines an inlet chamber portion 34 and an outlet chamber portion 36, which is sized smaller than the inlet chamber 30. Specifically, the inlet chamber portion 34 is sufficiently large for receiving the balls 28 when the RSV 10 is in the supine position (shown in FIG. 10). In that way, the balls 28 do not substantially obstruct the flow of CSF and therefore provide the low resistance path 16 through the inner shell 22. Put another way, the balls 28 sidestep the primary flow of CSF. Further, as shown in FIG. 11, the outlet chamber portion 36 is sufficiently small for receiving the balls 28 and placing the balls in position for substantially obstructing the flow of CSF and providing the high resistance path 18.

Moreover, each time the patient changes from the upright position to the supine position, the balls 28 randomly fall away, which acts as a self-cleaning mechanism should CFS debris be present. Each time the patient changes position the balls also reindex.

The small balls 28 are beneficial for their standard construction as they may be obtained from conventional sources. However, it is understood that the balls 28 can instead be custom made. Preferably, the balls 28 may be made from a lightweight, non-toxic, non-biodegradable material such as titanium or tantalum. The balls may also be made of a glass or jewel-type material, such as rubies or sapphires. The balls 28 further may be coated with a ceramic, plastic or other suitable materials to reduce their surface friction and aid in re-registering. For example, they may be Teflon-coated.

The shape of the inner and outer shells 20, 22 can be varied to accomplish an optimal flow pattern when the patient is supine and upright. For example, the chamber 30 could be conical. Also, the orifices 24, 26 could be varied in size, shape and number to achieve a desired flow rate in both the high and low resistive path configurations.

This construction is further beneficial because it does not include precision parts that need be assembled or milled. The shells 20, 22 can be either molded or easily formed in titanium using known methods. The orifices 24, 26 or slits, though small, should not require special technology to accomplish. For example, they could be formed by mechanical means or by laser cutting.

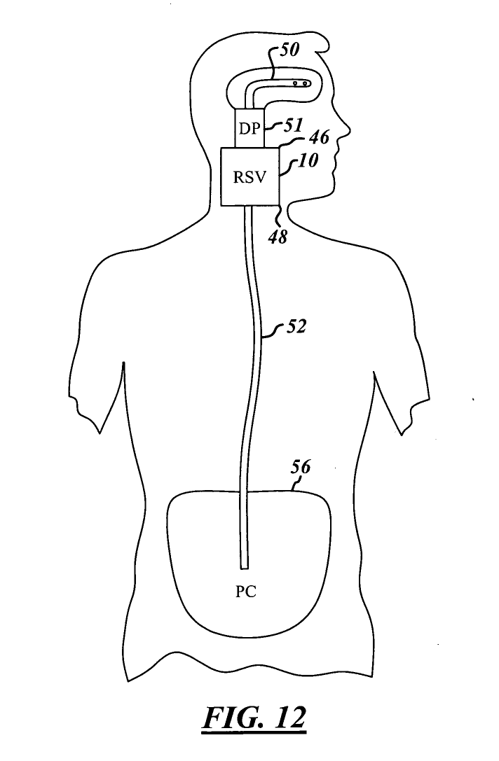

Referring now to FIG. 12, which illustrates one application of invention, the RSV 10 has a pair of opposing end portions 46, 48 which are operatively connected with an inlet catheter 50 and an outlet catheter 52. The inlet catheter 50 is within the fluid filled ventricle of the brain of a hydrocephalic patient. The RSV 10 may include a differential pressure valve 51 upstream of the direction of CSF flow. The differential pressure valve 51 can be of any known design. In such cases, the RSV acts as an overdrainage protection device in connection with the differential pressure valve 51. In this embodiment, the outlet catheter 52 is in fluid communication with the peritoneal cavity 56 of the patient. However, it is contemplated that the outlet catheter 52 can instead be in fluid communication with the atrium of the heart as desired. The outlet catheter 52 can be 1-3 feet in length, depending on the size of the patient.

While particular embodiments of the invention have been shown and described, it will be understood, of course, that the invention is not limited thereto since modifications may be made by those skilled in the art, particularly in light of the foregoing teachings. Accordingly, it is intended that the invention be limited only in terms of the appended claims.

What is claimed is:

1. A resistive shunt valve comprising:
a housing defining an axis and adapted for continuously conveying a flow of cerebrospinal fluid from an inlet to an outlet; and
a valve comprising at least one valve seat adjacent said outlet and at least two balls, said balls being the same size and adapted to register within a corresponding valve seat in the direction of cerebrospinal fluid flow,
wherein the number of balls is greater than the number of valve seats, and wherein each of said balls or seats includes an irregularity forming a leaky ball-in-seat seal, said valve adapted to prevent overdrainage of cerebrospinal fluid at a predetermined angle from a vertical orientation of said axis.

2. The resistive shunt valve of claim 1 comprising three valve seats and at least four balls.

3. The resistive shunt valve of claim 1 wherein the housing is a substantially straight-walled cylinder.

4. The resistive shunt valve of claim 1 wherein the housing is cylindrical and includes a widened body portion defined by an annular wall set at an angle with respect to said axis.

5. The resistive shunt valve of claim 4 wherein said angle is between 15 and 75 degrees from said axis.

6. The resistive shunt valve of claim 1 wherein said valve is movable between a clearing position and an obstructing position.

7. The resistive shunt valve of claim 1 wherein said inlet has a diameter less than a ball diameter.

8. The resistive shunt valve of claim 1 wherein said irregularity comprises balls having a non-spherical or scored surface.

9. The resistive shunt valve of claim 1 wherein said irregularity comprises valve seats having at least one notch or rib.

10. The resistive shunt valve of claim 1 wherein said irregularity comprises non-circular valve seats.

11. The resistive shunt valve of claim 1 wherein said balls occupy between approximately 20-50 percent of the housing volume.

12. The resistive shunt valve of claim 1 wherein a seat diameter (d) to ball diameter (D) ratio is between approximately 0.25-0.75.

13. A resistive shunt valve comprising:
a housing defining an axis and adapted for continuously conveying a flow of cerebrospinal fluid from an inlet to an outlet; and
a gravitational valve having one valve seat adjacent said outlet and at least two balls, one of said at least two balls adapted to register within said valve seat in the direction of cerebrospinal fluid flow, the valve creating a high resistance position for conveying said flow when said axis is in a vertical orientation, and a low resistance position for conveying said flow at a predetermined angle from said vertical orientation,
wherein said valve includes an irregularity forming a leaky seal providing a maximum flow rate of approximately 0.5 ml/min in said high resistance position.

14. The resistive shunt valve of claim 13 wherein the housing is a substantially straight-walled cylinder, and wherein said inlet has a diameter less than a ball diameter.

15. The resistive shunt valve of claim 13 wherein said irregularity comprises balls having a non-spherical or scored surface.

16. The resistive shunt valve of claim 13 wherein said irregularity comprises a valve seat having at least one notch or rib.

17. The resistive shunt valve of claim 13 wherein said irregularity comprises a non-circular valve seat.

18. The resistive shunt valve of claim 13 wherein said balls occupy between approximately 20-50 percent of the housing volume.

19. The resistive shunt valve of claim 13 wherein a seat diameter (d) to ball diameter (D) ratio is between approximately 0.25-0.75.

20. The resistive shunt valve of claim 13 wherein a ball adjacent said seat comprises a different material than said at least two balls.

21. The resistive shunt valve of claim 13 wherein all balls are the same size.

22. A resistive shunt valve for metering flow of cerebrospinal fluid, comprising:
a housing defining an axis and having an inlet and an outlet;
a plurality of balls, each the same size; and
at least two valve seats adjacent said outlet and each adapted to receive one of said plurality of balls in the direction of cerebrospinal fluid flow to form a leaky valve,
wherein the number of balls is greater than the number of valve seats, and
wherein each said leaky valve is adapted to provide a high resistance flow rate between a vertical orientation of said axis and a predetermined angle from said vertical orientation of said axis.

23. The resistive shunt valve of claim 22 wherein the housing is a substantially straight-walled cylinder, and wherein said inlet has a diameter less than a ball diameter.

24. The resistive shunt valve of claim 22 wherein said plurality of balls each have a non-spherical or scored surface.

25. The resistive shunt valve of claim 22 wherein each of said at least two valve seats comprise at least one notch or rib.

26. The resistive shunt valve of claim 22 wherein each of said at least two valve seats are non-circular.

27. The resistive shunt valve of claim 22 wherein said balls occupy between approximately 20-50 percent of the housing volume.

28. The resistive shunt valve of claim 22 wherein a seat diameter (d) to ball diameter (D) ratio is between approximately 0.25-0.75.
